(19) United States
(12) Patent Application Publication
(10) Pub. No.: US 2013/0245358 A1
(43) Pub. Date: Sep. 19, 2013

(54) CERVICAL BONE GROWTH STIMULATORS UTILIZING COMBINED MAGNETIC FIELD THERAPY

(71) Applicants: Jeffrey Johnson, Flowood, MS (US); John Lawrence Walker, Madison, MS (US); Joel Frank McCowan, Philadelphia, MS (US)

(72) Inventors: Jeffrey Johnson, Flowood, MS (US); John Lawrence Walker, Madison, MS (US); Joel Frank McCowan, Philadelphia, MS (US)

(21) Appl. No.: 13/790,576

(22) Filed: Mar. 8, 2013

Related U.S. Application Data
(60) Provisional application No. 61/610,231, filed on Mar. 13, 2012.

(51) Int. Cl. A61N 2/02

(52) U.S. Cl. CPC A61N 2/02
USPC 600/13

(57) ABSTRACT

Cervical bone growth stimulators utilizing combined magnetic field therapy is provided.

### CROSS-REFERENCE TO RELATED APPLICATIONS

This application claims priority to U.S. Provisional Application No. 61/610,231 filed on Mar. 13, 2012, which is incorporated by reference herein.

### TECHNICAL FIELD

The present invention relates to cervical bone growth stimulators utilizing combined magnetic field therapy.

### BACKGROUND

Electrical bone growth stimulators are a supplemental form of therapy to enhance the body's orthopedic healing process. Based on the surgeon's preference, or if the patient has risk factors for fusion, an electrical bone growth stimulator may be used as an adjunct to spinal fusion surgery to help enhance the chances of achieving a successful bone fusion.

Bone stimulators can either be implanted under the skin (internal) or worn on the outside of the skin (external). Since external bone growth stimulation devices are worn outside the skin, they do not require surgical implantation or extraction. Typically, the device is worn after spine fusion either as:

small electrodes that are placed directly over the fusion site and deliver Capacitive Coupling (CC) or; one or two treatment coil(s) placed into a brace or directly onto the skin that deliver an electromagnetic field such as a Pulsed ElectroMagnetic Field (PEMF) or a Combined Magnetic Field (CMF).

Unlike an internal (implanted) bone growth stimulator, an external bone growth stimulator may also be prescribed for the patient to use several weeks or months after the fusion surgery if the bone is not fusing as desired,

A potential advantage of external stimulators versus internal stimulators is that the external device is usually a less expensive treatment option and does not require the potential second surgery to remove the battery pack. Another advantage is that an external bone stimulator can be added after the fusion has been done if there is concern that the bone graft is not healing and a nonunion is developing.

An external electrical stimulator is usually lightweight and powered by a battery, so it is very portable. There is still a need in the art for a bone growth stimulator that is capable of treating the cervical spinal region effectively following cervical spinal fusion surgery.

### SUMMARY

In an embodiment, the present invention provides a cervical bone growth stimulation system. The system includes a transducer assembly comprising at least one transducer coil that generates a combined magnetic field to a cervical region of a patient's vertebral column to stimulate bone growth in the cervical region. The system also includes a control unit programmed to drive a CMF output signal to the at least one transducer coil to stimulate bone growth of the cervical region. Preferably, the transducer assembly includes a substantially convex, oval-shaped, rigid outer housing that contains a similarly shaped single transducer coil.

### BRIEF DESCRIPTION OF THE DRAWINGS

FIG. 1 is an isometric view of an embodiment of a device according to the present invention.

FIG. 2 is a top view of the embodiment depicted in FIG. 1.

FIG. 3 is a front view of the embodiment depicted in FIG. 1.

FIG. 4 is a side view of the embodiment depicted in FIG. 1.

### DETAILED DESCRIPTION

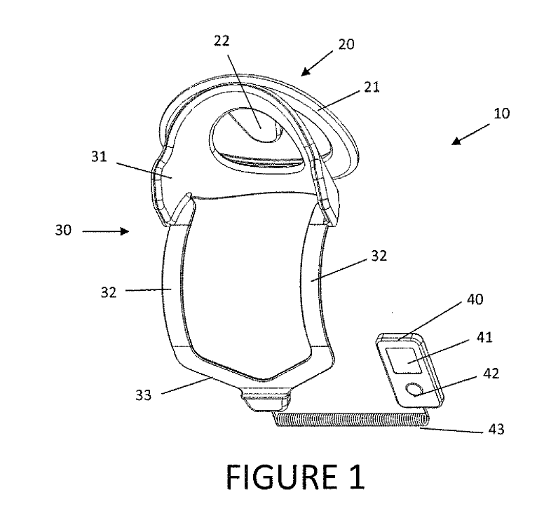

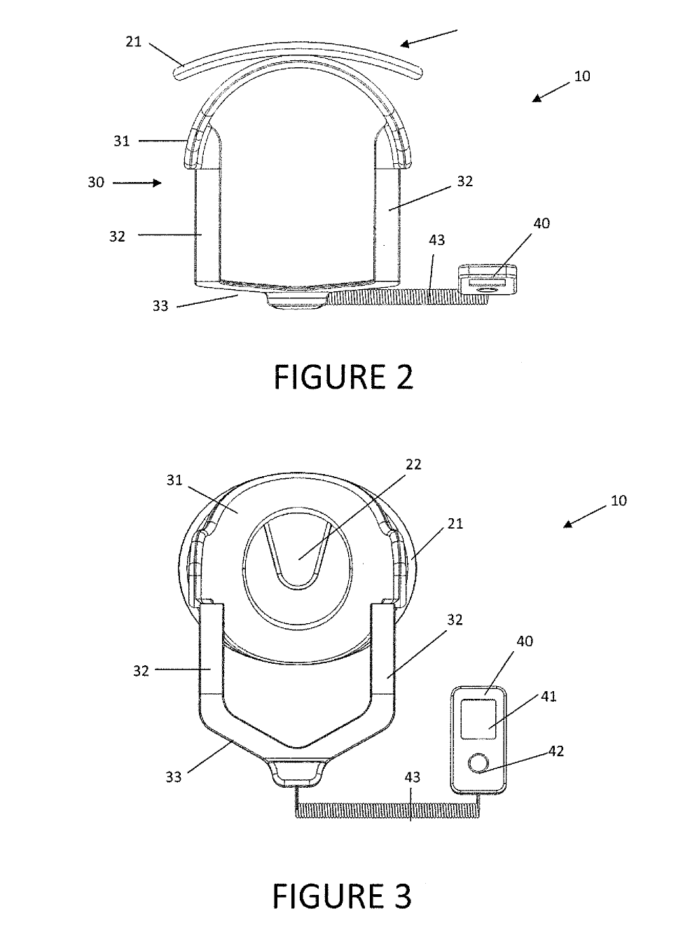

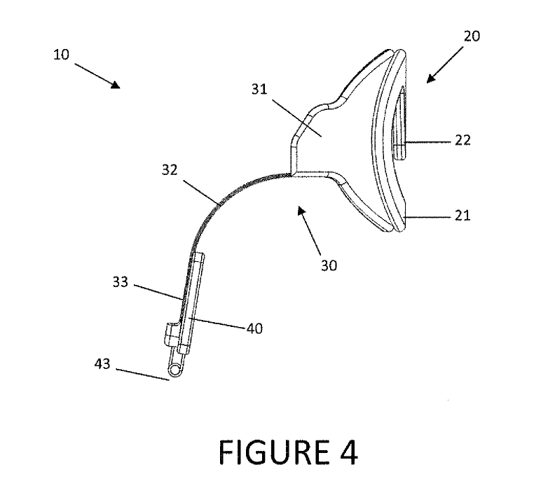

The present invention provides bone growth stimulators, such as cervical bone growth stimulators utilizing CMF therapy. FIGS. 1-4 show an exemplary cervical bone growth stimulator device 10 that can be used to stimulate bone growth in the cervical spinal region. In certain embodiments, device 10 works by delivering a magnetic field therapy through a collar-mounted transducer assembly 20. Specifically, the treatment delivered by device 10 can be CMF therapy, which comprises an alternating magnetic field superimposed over a static magnetic field. CMF therapy uses extremely low frequency magnetic fields that are functionally dependent upon ion cyclotron resonance frequencies as described in “The Charge-to-Mass ICR Signature in Weak ELF Bioelectromagnetic Effects” by Abraham R. Liboff, which is incorporated by reference herein.

In an embodiment, device 10 includes a transducer assembly 20 comprising a substantially convex, oval-shaped outer housing 21, and a hollowed arm 22. By “substantially” is meant that the shape of the described component does not need to have the mathematically exact described shape, such as convex or oval, but can have a shape that is recognizable by one skilled in the art as generally or approximately having the described shape, such as convex or oval. In a preferred embodiment, the arm is a single arm. Preferably, the housing of the transducer assembly is rigid. The convex, oval-shaped outer housing 21 preferably contains a similarly shaped transducer coil (not shown), and more preferably a similarly shaped single transducer coil, which is used to deliver the CMF therapy. The transducer coil receives its drive signal from the control unit 40 over a multi-conductor cable 43 or wirelessly. The convex, oval shape of the outer housing 21 is such that it preferably matches the natural shape of a user's cervical spinal region; however, outer housing 21 and the transducer coil could be made in other shapes, for example, a circle or a square. Outer housing 21 also need not be convex, but could be flat or concave. In other embodiments, the transducer assembly 20 contains multiple transducer coils. In yet other embodiments, the transducer coil can be encased within a neck band 31, described in more detail below.

Preferably, arm 22 is an inner single arm that more preferably extends to the center of the transducer assembly 20, and contains a magnetic sensor (not shown), which provides feedback to the control unit 40 so that the magnitude of the CMF therapy delivered to the transducer coil can be precisely controlled. This feedback can transmitted to the control unit 40 over the multi-conductor cable 43. Typically, the transducer assembly 20 can be made of plastic, but various types of materials can be used in order to produce a rigid and lightweight assembly. In other embodiments, the feedback and/or coil drive signals may be transmitted wirelessly, eliminating the need for a multi-conductor cable 43. In yet other embodiments, the magnetic sensor can be encased within neck band 31.

In certain embodiments, device 10 includes a separately carried, handheld control unit 40 that is connected to the transducer assembly 20 by multi-conductor cable 43. Multi-conductor cable 43 can be routed to the transducer assembly 20 by entering the collar assembly 30 at the abdominal strap 33 and traversing the left or right chest strap 32 and the neck band 31. At the neck band 31, the multi-conductor cable 43 can exit the collar assembly 30 and enter the transducer assembly 20. In another embodiment, the multi-conductor cable 43 could be attached to the outside of the collar assembly 30, using various attachment methods such as Velcro or snaps. In yet another embodiment, the multi-conductor cable 43 could connect directly to the transducer assembly 20, without being embedded in, or attached to the collar assembly 30. It should also be recognized that there are numerous other methods in which the multi-conductor cable 43 could be routed from the control unit 40 to the transducer assembly 20. The handheld control unit 40 could also mount onto the collar assembly 30 using a number of methods, including but not limited to, a belt clip, Velcro, or magnets.

Within control unit 40 resides a processor such as a printed circuit board that is energized by a power source, which could include primary or secondary batteries, or which could be obtained from an electrical outlet, The processor preferably includes both the drive and control electronics integrated such that they are both contained within control unit 40. The drive electronics produce the desired CMF output signal, and send them to the transducer coil, which can be enclosed in transducer assembly 20. The control electronics can provide system status information to the user through the LCD display 41, and command the drive signal according to a firmware program residing within the control unit 40. A pushbutton 42 can also be included on the control unit 40 so that the user may command various system functions of the device 10. The drive electronics need not be located in the control unit 40, but could be placed anywhere on the device 10, including in the collar assembly 30 or in the transducer assembly 20. In another embodiment, the control unit 40 could be integrated directly into the collar assembly 30, such that a single, integrated device would be realized. And in yet another embodiment, the collar assembly 30 may include a dock, such that the control unit 40 may plug directly into the collar assembly 30, allowing the user to choose whether to operate the device 10 as a single, integrated unit, or with a separate, handheld control unit 40.

In certain embodiments, device 10 includes a collar assembly 30 that is worn around the user's neck, either with or without clothing, or a neck brace. In certain embodiments, the collar assembly 30 comprises neck band 31, chest straps 32, and an abdominal strap 33. Neck band 31 is preferably constructed of a soft, flexible material that naturally conforms to the shape of the wearer's neck. The neck band 31 is not semi-rigid, and therefore cannot be formed into, and retain a desired shape. The neck band 31 secures the transducer assembly 20 by using Velcro straps (not shown), or some other securing means like snaps or buckles. The chest straps 32 preferably connect directly to the neck band 31 on one end, and on the other end connect to the abdominal strap 33. The chest straps 32 may attach to the abdominal strap 33 using Velcro, snaps, buckles, or any other method of attachment. The center of the abdominal strap 33 can be weighted such that device 10 is held securely in place during use. In other embodiments, the abdominal strap 33 and weights could be removed or reconfigured such that the transducer assembly 20 and neck band 31 rests securely in place. In yet other embodiments, the neck band 31 could be secured in place by utilizing other suitable configurations such as, for example, neck straps, head straps, back straps, or shoulder straps.

The foregoing description and examples have been set forth merely to illustrate the invention and are not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

1. A cervical bone growth stimulation system comprising:
a transducer assembly comprising at least one transducer coil that generates a combined magnetic field to a cervical region of a patient's vertebral column to stimulate bone growth in the cervical region; and
a control unit programmed to drive a CMF output signal to the at least one transducer coil to stimulate bone growth of the cervical region.

2. The system of claim 1, wherein the transducer assembly comprises a substantially convex, oval shaped housing encasing the at least one transducer coil.

3. The system of claim 1, wherein the at least one transducer coil has a configuration similar to the configuration of the housing.

4. The system of claim 2, wherein the housing matches the natural shape of a cervical spine region.

5. The system of claim 1, wherein the at least one transducer coil is a single transducer coil.

6. The system of claim 1, wherein the at least one transducer coil is a plurality of transducer coils.

7. The system of claim 1, wherein the control unit delivers the CMF output signal to the at least one transducer coil via a conductor cable.

8. The system of claim 1, wherein the housing comprises a hollowed ring and an arm extending caudally from the ring.

9. The system of claim 8, further comprising a magnetic sensor encased by the arm, the magnetic sensor providing a feedback signal to the control unit, the control unit controlling the magnitude of the CMF output signal in response to the feedback signal.

10. The system of claim 1, further comprising a collar assembly comprising a neck band connected to the transducer assembly, a chest strap connected to the neck band, and an abdominal strap connected to the chest strap.

11. The device of claim 10, further comprising a magnetic sensor encased within the neck band.

12. The system of claim 1, wherein the control unit comprises circuitry comprising drive and control electronics, the drive electronics producing a desired CMF output signal, the control electronics providing system status information and commanding the drive signal according to firmware program residing within the control unit.

13. The system of claim 1, further comprising drive electronics that produce the desired CMF output signal.

14. The system of claim 13, wherein the drive electronics are located in the collar assembly or the transducer assembly.

15. The system of claim 1, wherein the control unit is integrated in the collar assembly.

16. The system of claim 1, further comprising a dock in the collar assembly allowing the control unit to plug directly into the collar assembly.
